(19) United States
(12) Patent Application Publication
(10) Pub. No.: US 2025/0331974 A1
(43) Pub. Date: Oct. 30, 2025

(54) Mandibular Anesthesia Curved Dental Needle

(71) Applicant: Jelani T. Washington, Conyers, GA (US)

(72) Inventor: Jelani T. Washington, Conyers, GA (US)

(21) Appl. No.: 18/644,918

(22) Filed: Apr. 24, 2024

(51) Int. Cl. A61C 19/08; A61M 19/00

(52) U.S. Cl. CPC A61C 19/08; A61M 19/00

(57) ABSTRACT

The present invention provides a curved needle design which deflects to an advantageous angle when inserted within the medial posterior soft tissue of the patient lower jaw. The deflected needle tip is more perpendicular to the vertical wall of the posterior ramus and promotes the needle tip contacting bone proximate to the inferior alveolar nerve prior to injection of anesthetic. The needle design also improves the tactile feedback to the dental practitioner as the needle penetrates tissue and contacts bone.

### CROSS-REFERENCE TO RELATED APPLICATION

This application is a continuation in part of U.S. Utility application Ser. No. 16/848,731 filed on Apr. 14, 2020, the entirety of which is incorporated herein by reference.

### REFERENCES CITED

### Other Publications

1. De St Georges J. How dentists are judged by patients. Dent Today 2004; 23 (8): 96-99.
2. Claffey E, Reader A, Nusstein J, Beck M, Weaver J. Anesthetic efficacy of articaine for inferior alveolar nerve blocks in patients with irreversible pulpitis. JOE. 2004; 30:568-571.
3. Levy T. P. An assessment of the Gow-Gates mandibular block for third molar surgery. J AM Dent Assoc 1981; 103:37-41.
4. Robertson W E. Clinical evaluation of mandibular conduction anesthesia. Gen Dent 1979; 27:49-51.
5. Montagnese T, Reader A, Melfi R. A Comparative study of the Gow-Gates technique and a standard technique for mandibular anesthesia. JOE 1984; 10:158-163.
6. Quinn C L. Injection to anesthetize the difficult tooth. J Calif Dent Assoc. 1998; 26:665-667.
7. Bassett K B, DiMarco A C, Naughton D K. Local Anesthesia for Dental Professionals. 2nd ed. Upper. Saddle River, NJ. Pearson Prentice Hall: 2014.
8. Malamed S F. Handbook of Local Anesthesia. 6th ed. St. Louis, Mo. Elsevier Mosby: 2013.
9. Evers H, Haegerstam G. Introduction to Dental Local Anesthesia. 2nd ed. Fribourg, Switzerland: Mediaglobe SA; 2003.
10. Manski M C, Cartee D L. Tips for administering inferior alveolar nerve block. Dimensions of Dental Hygiene. 2018: 13 (11); 60-63.
11. Bassett, K. B. (2010). Local Anesthesia for Dental Professionals (pp. 330). Pearson Education, Inc.
12. Malamed, S. G. (2004). Handbook of local anesthesia (5th ed., pp. 227-253). St. Louis: Elsevier Mosby.
13. Delgado-Molina E, Tamarit-Borras M, Berini-Ayetes L, Gay-Escoda C. Comparative study of two needle models in terms of deflection during inferior alveolar nerve block. Med Oral Patol Oral Cir Bucal. 2009 Sep. 1; 14 (9): e440-4.
14. Kenedy S, Reader A, Nusstein J, Beck M, Weaver J. The significance of needle deflection in success of the inferior alveolar nerve block in patients with irreversible pulpitis. JOE. 203; 29:630-633.
15. Haas D A, Alternative mandibular nerve lock techniques: A review of the Gow-Gates and Akinosi-Vazirani closed-mouth mandibular nerve block techniques. JADA. 2011; 142 (9 suppl): 8S-12S.
16. Reed K, Malamed S, Fonner. Local Anesthesia Part 2: Technical Considerations. Anesth Prog. 2012; 59:127-137.
17. Shahi S, Rahimi S, Yavari H R, Ghasemi N, Ahmadi F. Success rate of 3 injection methods with articaine for mandibular first molars with symptomatic irreversible pulpitis: A consort randomized double-blind clinical trial. J OE. 2018; 44:1462-1466.
18. Palti D G, de Almeida C M, Rodrigues A, Andreo J C, Oliveira J E. Anesthetic technique for inferior alveolar nerve block: a new approach. J Appl Oral Sci. 2011; 19 (1): 11-15.

### BACKGROUND OF THE INVENTION

### 1. Field of the Invention

The present invention relates generally to needles used in the injection of pharmaceutical drugs during dental procedures. More specifically, the present invention has effective application in the lower jaw injection of mandibular anesthesia during dental procedures.

### 2. Description of the Related Art

Sound local anesthesia technique is a corner-stone of dentistry. Some of the most common statements heard are: “Do I have to get a shot?” or “Just don't show me the needle!” or “I'm fine after the needle.” De St Georges, an internationally renowned dental practice management speaker and author, surveyed her audiences on how they perceived dentists 1. She identified 20 key areas. On that list, the second most important area of concern was that the procedure “does not hurt” 1. The most important area of concern was “a painless injection” 1. When you consider comments by patients, the two most common areas of concern involve pain. Therefore, the importance of sound anesthesia administration technique and accuracy cannot be underestimated.

As depicted in FIG. 1, a particular problematic injection site is to the mandible (lower jaw 10). Mandibular anesthesia success rates have been shown to range from as low as 19% to as high as 98%²⁻⁶ depending on the criteria for success. This wide range of dental anesthesia success suggests there is a degree of ambiguity with the technique and/or a deficiency in instrument design used to attempt mandibular anesthesia. The most common technique taught is the Halstead technique. As the mandibular nerve descends from the Trigeminal ganglion it makes several branches, one of which is the inferior alveolar nerve 12 (primary nerve of the lower jaw). It is the inferior alveolar nerve that is anesthetized before it enters the Mandibular foramen 14. In the Halstead technique, a syringe needle tip is used to gain proximity to the mandibular foramen 14 where the inferior alveolar nerve enters the mandible. Also depicted in FIG. 1, are the caronoid notch 16, lingula 18 and internal oblique ridge 20 (boney wall anterior to the opening into the lower jaw for the primary nerve of the lower jaw).

As depicted in FIGS. 2A and 2B, the injection site 24 of the Halstead technique is performed by the dental practitioner by locating several physical landmarks including the coronoid notch 16 (depression along the anterior surface of the ramus), and the lateral depression of the pterygomandibular raphe 22. Also depicted in FIG. 2B are the facial nerve 26, lingual nerve 28, medial surface of the ramus mandible 30, and the masseter muscle 32.

The straight beveled-needle is the most widely used needle for administering anesthetics for a mandibular block. The bevel at the tip of the needle allows for deflection (14). However, the resulting deflection often results in a missed block or at the bare minimum a need to adjust by re-inserting the needle causing more patient discomfort. There are several devices used to administer dental anesthetics, all of which use some form of a straight beveled-needle. Whether the mandibular block is performed with a standard dental anesthetic syringe (14), or as a Leur-Lok type needle attached to a safety needle, or a computer controlled injection system such as The Wand (14), the resulting deficiencies are still present because of the straight beveled-needle design.

As further depicted in FIGS. 3, 4A, 4B, and 4C once the landmarks are identified, the syringe barrel 40 is oriented over the opposite side premolars 34 (teeth between molar and canine teeth) at an angle parallel to and above the mandibular molar occlusal plane 36 (imaginary plane formed by the chewing surfaces of adjacent teeth), and the beveled tip 44 of a 25 or 27 gauge, straight 35 mm needle 42 is inserted in the mucous membrane lateral (next to, away from the midline, the pterygomandibular raphe) to the pterygomandibular raphe until it contacts bone directly over the nerve as it enters the mandibular foramen (opening of the lower jaw bone for the primary nerve of the lower jaw), at injection site 24⁷⁻¹⁰. As depicted in FIGS. 4B-4C, failure to contact bone is often related to mandibular ramus flare (the external rotation of the posterior vertical wall of the lower jaw) and then requires re-orientation of the needle.

The objective of the needle insertion is to deposit anesthetic in close proximity to the inferior alveolar nerve to provide nerve anesthesia to the teeth and soft tissue along the nerve's path of innervation. A common problem, however, is the aforementioned wide range of anesthetic success. The Halstead technique makes several assumptions: there are contralateral premolar teeth (opposite side teeth), there is a definitive ipsilateral occlusal table (same side chewing surface), the ramus does not flare, the opening in the mandible for the nerve is in a consistent location and that the practitioner can identify the boney and soft tissue physical landmarks.

As depicted in FIGS. 4A, 4B, and 4C, when using a straight, beveled-needle, depending on the flare of the ramus, the needle may or may not contact bone. If the needle contacts bone prematurely as depicted in FIG. 4A, or does not contact bone at all as depicted in FIG. 4B, 4C, the anesthetic may not be deposited close enough to the mandibular foramen. At that point, the needle is retracted, the angle of needle insertion is adjusted, and the needle re-inserted. Often, the needle is deflected away from the intended area of deposition. As a result of this deflection, the chance of successful anesthesia injection is reduced. The wide range of success can be attributed to multiple factors including: anatomical variations¹¹, volume of anesthetic¹¹, technique proficiency, and needle deflection^{12, 13, 14}, just to name a few.

The deficiency in predictability has brought about a need for alternative techniques to achieve mandibular anesthesia. For example: the Gow-Gates technique^{5, 15, 16}, the Vazirani-Akinosi technique^{15, 16}, and modifications to the traditional Halstead technique^{17, 18}. However, these various techniques also present the same challenges that may encourage a range of success. Until now, attempts to improve the predictability of mandibular anesthesia have been centered around the technique. The instrument used to deliver the anesthetic, the needle, should be addressed.

Needle deflection plays a key role when considering problems with inadequate dental anesthesia injection and should be considered an important aspect in how a dental needle is used during the delivery of anesthetic. As depicted in FIG. 4, once a needle is inserted into soft tissues of the oral cavity, and particularly when considering an inferior alveolar (primary nerve of the lower jaw) nerve block, depending on the physical landmarks encountered or lack-there-of, the needle is often adjusted to compensate and will deflect towards or away from the intended site of injection. This deflection makes a significant contribution to the variability in anesthetic success. In practice, the more significant the needle angle adjustment, the more significant the deflection and variability in the actual anesthetic injection location.

Accordingly, what is needed in the art is a needle designed to actively influence needle deflection towards the intended site of anesthetic deposition. The needle design should promote injection of the anesthetic proximate to the nerve without reposition or adjustment of the needle injection angle required. It is thus to such a needle design that the present invention is primarily directed.

### SUMMARY OF THE INVENTION

The disadvantages of the prior art are overcome by the present invention which, in one aspect is dental procedure for injection of pharmaceutical compound into an injection site at a posterior vertical wall of a patient mouth during an inferior alveolar nerve block performed by a clinician. The patients mouth having a internal oblique ridge, a pterygomandibular raphe, a lingula, a mandible bone, and a inferior alveolar nerve. The procedure utilizing a needle assembly affixed to a syringe. The needle includes a needle shaft, a needle hub, a needle tip, with a central bore passing thru each. The syringe includes a syringe barrel and a plunger.

The procedure steps include the clinician palpating the patient's internal oblique ridge using the clinician's thumb or index finger, the internal oblique ridge providing a landmark for identification of an injection site. Identifying the injection site as medial to the internal oblique ridge and lateral to the pterygomandibular raphe and directly adjacent and centered to the palpating thumb or index finger. Inserting the needle tip at the identified injection site by applying an axial force to the syringe barrel. The needle shaft comprising a centerline axis and a curve therein such that the centerline axis of the needle shaft at the tip is at an angle when compared to the centerline axis of the needle shaft at the needle hub.

Wherein the centerline axis of the needle shaft at the needle shaft tip is deflected towards the lingula of the patient by the axial force. The clinician applying axial force to the syringe barrel until the needle tip contacts the bone of the mandible. The clinician withdrawing the needle tip 1 to 2 millimeters and injecting the pharmaceutical compound adjacent the inferior alveolar nerve.

In an alternative embodiment of the procedure of the present invention, wherein after withdrawing the needle tip 1 to 2 millimeters, the clinician retracts the syringe plunger to ensure he or she is not injecting the pharmaceutical compound into a blood vessel. And upon verifying no blood is present, the pharmaceutical compound is injected. If blood is present, the needle tip is withdrawn an additional 1 millimeter. The clinician again retracts the syringe plunger, and upon verifying no blood is present, the pharmaceutical compound is injected.

In other alternative embodiments of the procedure of the present invention the pharmaceutical compound is an anesthetic. As the needle tip is inserted into the injection site the angled needle shaft centerline axis facilitates access to the injection site by avoiding physical obstacles presented by the patient anatomy to the needle assembly and syringe barrel.

In other alternative embodiments of the procedure of the present the angle between the centerline axis of the needle shaft at the tip when compared to the centerline axis of the needle shaft at the hub is between 15 and 45 degrees. The angle between the centerline axis of the needle shaft at the tip when compared to the centerline axis of the needle shaft at the hub is substantially 30 degrees. The needle shaft used in the procedure has a length between 20 millimeters to 40 millimeters from tip to hub measured along the needle shaft centerline. The needle shaft used in the procedure comprises a central bore between 20 and 32 gauge therein for passing pharmaceutical chemicals there thru. The needle shaft used in the procedure comprises a central bore between 25 and 27 gauge therein for passing pharmaceutical chemicals there thru.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE DRAWINGS

FIG. 1 is a side-perspective view of the human lower jaw.

FIG. 2A is a side view of the human mouth with landmarks used in the injection of anesthesia.

FIG. 2B is a cutaway depiction of the lower jaw with anesthesia injection needle of the prior art.

FIG. 3 is a side perspective view of one side of the lower jaw.

FIGS. 4A, 4B, 4C are perspective views of the lower jaw depicting anesthesia injection by a prior art needle.

FIG. 5 is a side view of the components of the angled needle of the present invention.

FIGS. 6A, 6B, 6C are perspective views of the lower jaw depicting anesthesia injection by the needle of the present invention.

FIG. 7 is a top perspective depiction of anesthesia injection using the angled needle of the present invention.

FIG. 8A is a side perspective view of the human mouth with landmarks used in the injection of anesthesia with the present invention.

FIG. 8B is a side perspective view of the human mouth with anesthesia injection by the needle of the present invention.

FIG. 9A is a side perspective view of a prior art needle tip.

FIGS. 9B, 9C, and 9D are side perspective views of embodiments of the needle tip of the present invention.

### DETAILED DESCRIPTION OF THE INVENTION

The present invention provides a curved needle design which deflects to an advantageous angle when inserted within the medial posterior soft tissue of the patient's lower jaw. The deflected needle tip is more perpendicular to the vertical wall of the posterior ramus and promotes the needle tip contacting bone proximate to the inferior alveolar nerve prior to injection of anesthetic. The needle design also improves the tactile feedback to the dental practitioner as the needle penetrates tissue and contacts bone.

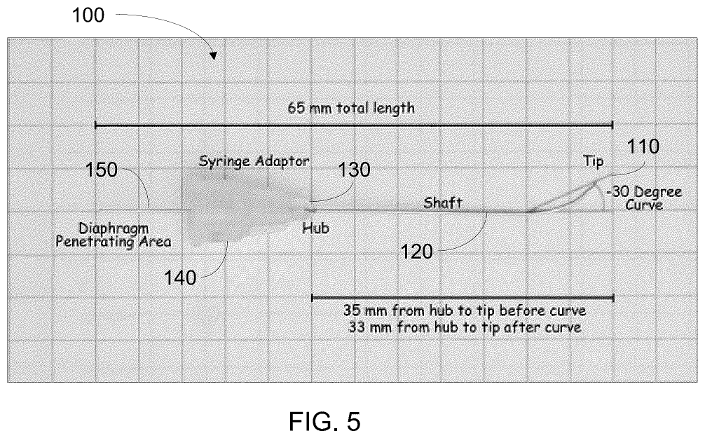

As depicted in FIG. 5, the dental anesthetic needle assembly of the present invention is composed of a continuous length of stainless steel. There are five key features/components of the needle assembly 100 of the present invention. The needle assembly includes the tip 110, the shaft 120, the hub 130, syringe adaptor 140 and, the diaphragm penetrating area 150.

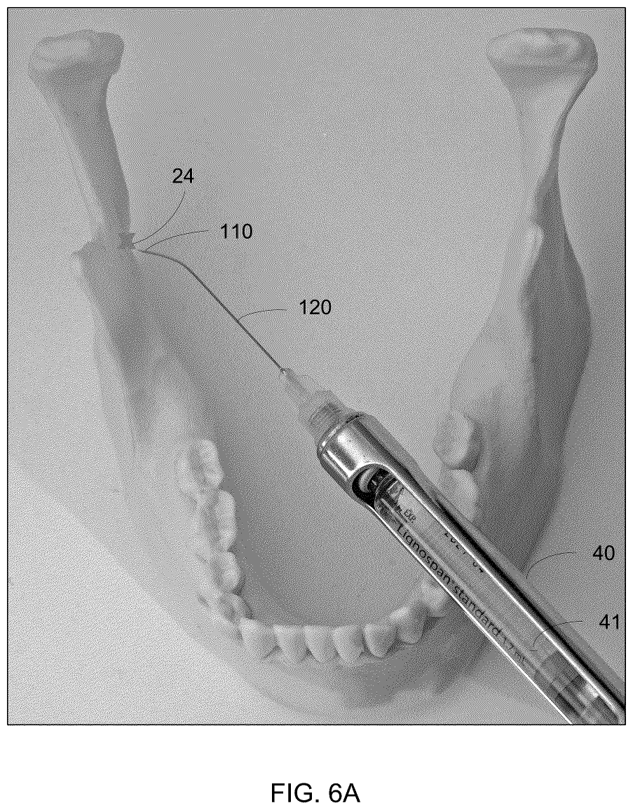

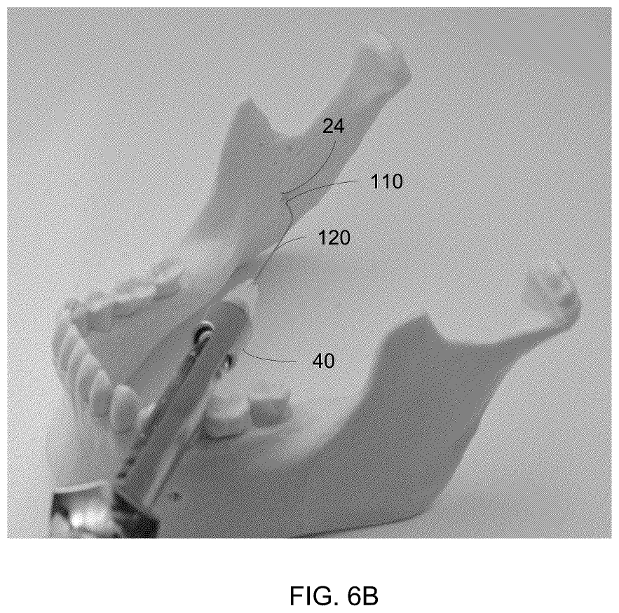

As further depicted in FIG. 5, the portion of the shaft 120 proximate to the tip 110 that will penetrate soft tissue is curved to a preferable 30 degree angle. The curve of the shaft 120 may be continuous along the shaft between the hub 130 and tip 110 to end in a tip angled in relation to the centerline of the shaft at the hub, or the curve may be formed by a more abrupt small radius bend along the length of the shaft 120, or any combination thereof. The curve in the needle shaft 120 is accomplished without collapsing or substantially reducing the internal bore of the needle thru which anesthesia or other pharmaceutical compounds are passed. In a first embodiment depicted in FIGS. 5, 6A, 6B, and 6C, the shaft 120 has a substantially straight portion adjacent the hub 130, and a substantially straight portion adjacent the tip 110, with a curve there-between to form an angle between the centerline axis of the shaft at the tip, and the centerline axis of the shaft at the hub. In other alternative embodiments, the angle formed between the centerline axis of the shaft 120 at the tip 110, and the centerline axis of the shaft 120 at the hub 130 may be between 15 and 45 degrees.

In the first embodiment, the shaft measures a length of thirty-five millimeters from tip 110 to hub 130 measured along the shaft 120 centerline. The shaft 120 has a twenty-five or twenty-seven gauge internal diameter and 0.4 to 0.5 millimeter outer diameter. As will be appreciated by those skilled in the art, thirty and thirty two millimeter shafts are also considered long needles, whereas twenty and twenty-five millimeter shafts are also considered short needles. In alternative embodiments, the shaft may be between 20 millimeters to 40 millimeters in length, and the shaft may have an internal gauge between and including 20 to 32. In other alternative embodiments, the internal needle gauge and/or the needle outer diameter may vary along the needle length.

As depicted in FIGS. 5, 6A, the hub 130 is the component that connects the shaft 120 to the syringe adaptor 140 via adhesive or a press fit in the plastic hub. The plastic syringe adaptor 140 has an internal thread pattern to affix the needle to the syringe barrel. Through the syringe adaptor continues the remaining twenty-five millimeters of the stainless steel needle shaft 150 that penetrates the diaphragm of the anesthetic carpule 41. As appreciated by those skilled in the art, in other applications of the present invention the carpule 41 may be filled with other pharmaceutical compounds. As further appreciated by those skilled in the art, the curved needle design of the present invention may be used with other syringe designs, for example single use disposable syringes. The needle assembly may also engage the syringe via a press fit.

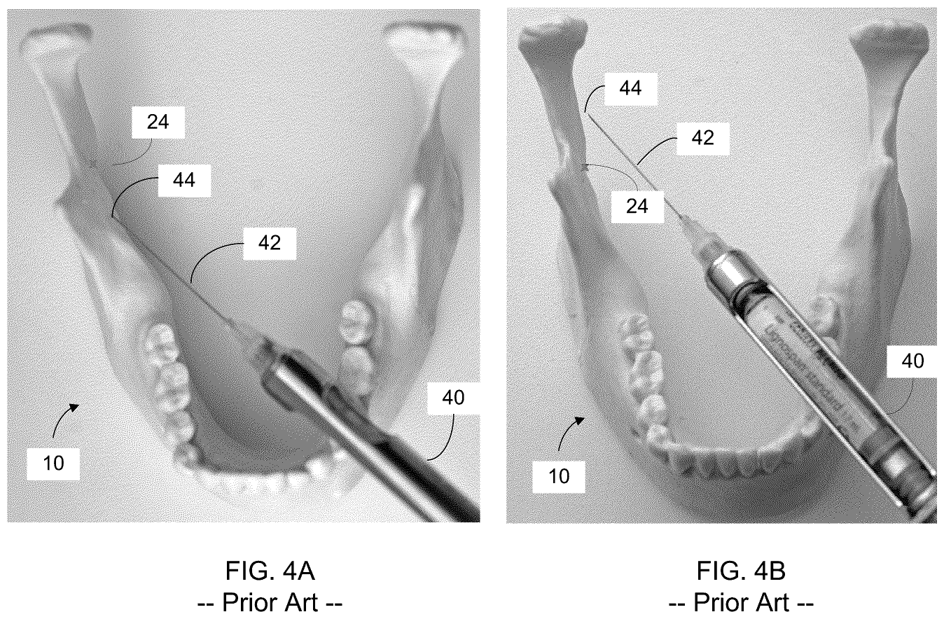

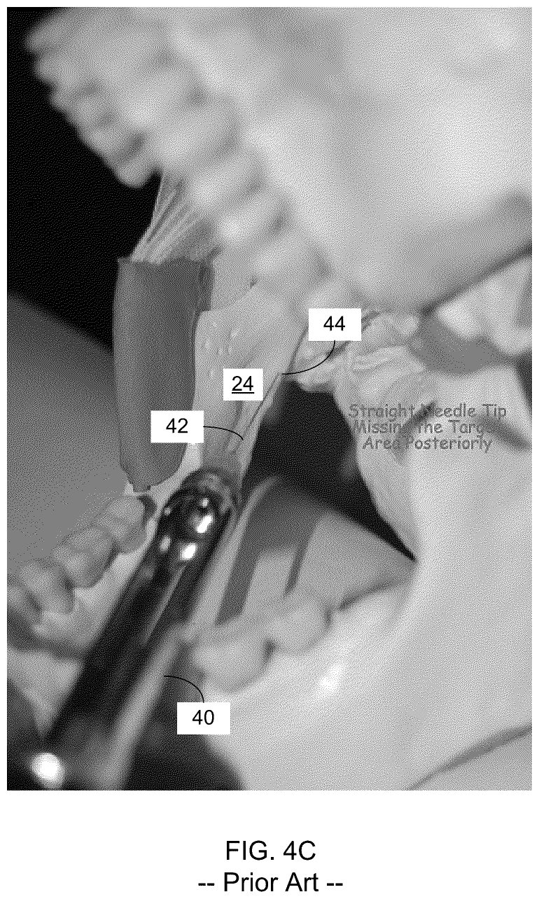

The dental procedure technique of using the needle assembly of the present invention differs over the prior art Halstead technique. As depicted in FIGS. 6A, 6B and 6C, the 30 degree curvature at the tissue penetrating end, tip 110, of the curved needle 100 is incorporated to exploit the concept of needle deflection. The curved or angled needle shaft 120 reduces the variability of the procedure due to anatomical differences between patients, particularly as it relates to mandibular ramus flare, i.e. the external rotation of the posterior vertical wall of the lower jaw. Once the physical landmarks in the patients mouth are identified, the syringe barrel 40 is oriented over the opposite side premolars 34 (teeth between molar and canine teeth) at an angle parallel to and above the mandibular molar occlusal plane (imaginary plane formed by the chewing surfaces of adjacent teeth), and the beveled tip 110 of the curved needle 100 of the present invention is inserted in the mucous membrane lateral (next to, away from the midline, the pterygomandibular raphe) to the pterygomandibular raphe until it contacts bone proximate to the nerve as it enters the mandibular foramen (opening of the lower jaw bone for the primary nerve of the lower jaw), at injection site 24⁷⁻¹⁰. As depicted in FIG. 6C, the curved needle shaft 120 biases the needle tip 110 deflection towards the lingula and mandibular foramen. The curved needle 120 design of the present invention also improves the tactile feedback to the practitioner as the needle penetrates and/or pierces tissue and contacts bone. Referring back to prior art FIGS. 4A, 4B and 4C, a straight needle 42 is more likely to miss the anesthesia target area either anteriorly or posteriorly.

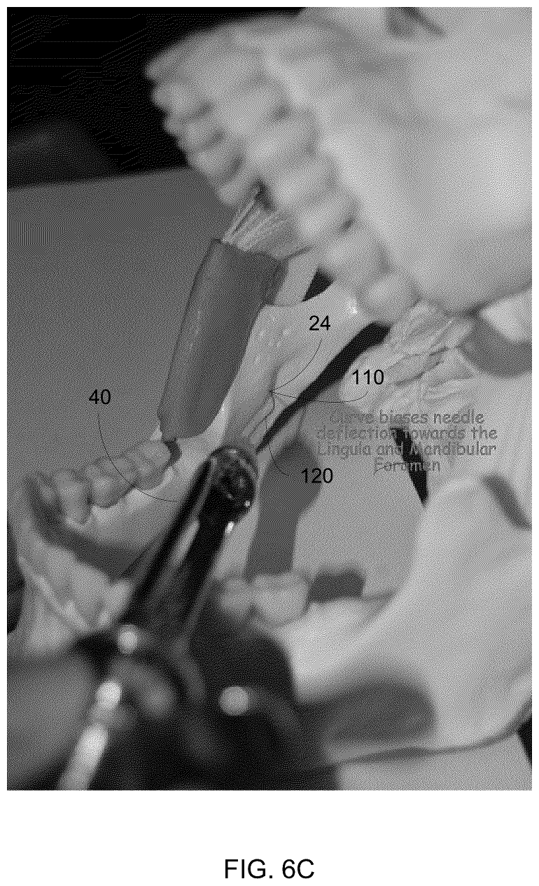

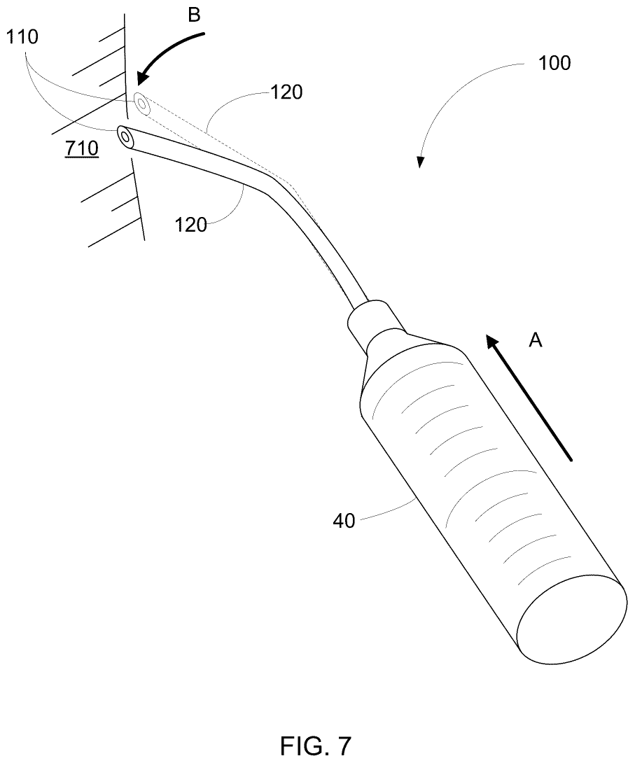

As depicted in FIG. 7, the curved needle design 100 of the present invention deflects the tip 110 of the needle shaft 120 to be more perpendicular to the posterior vertical wall 710. As pressure is applied axially to the syringe body in the direction of Arrow “A”, the centerline axis at the tip 110 of the curved needle shaft 120 is deflected to be more perpendicular to the posterior vertical wall 710 as depicted by curved Arrow “B”. Stated another way, the insertion force axially down the center of the syringe body supplied by the dental practitioner is opposed by the offset needle tip 110 and results in a bending moment applied to the needle shaft 120. The bending moment deflects the centerline axis of the needle shaft 120 at the needle tip 110 to be more perpendicular to the posterior vertical wall 710.

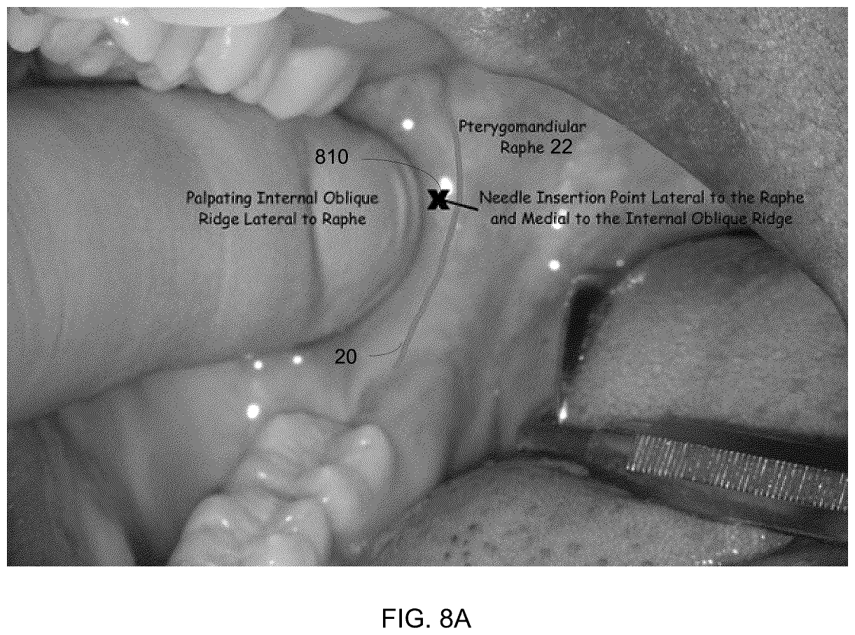

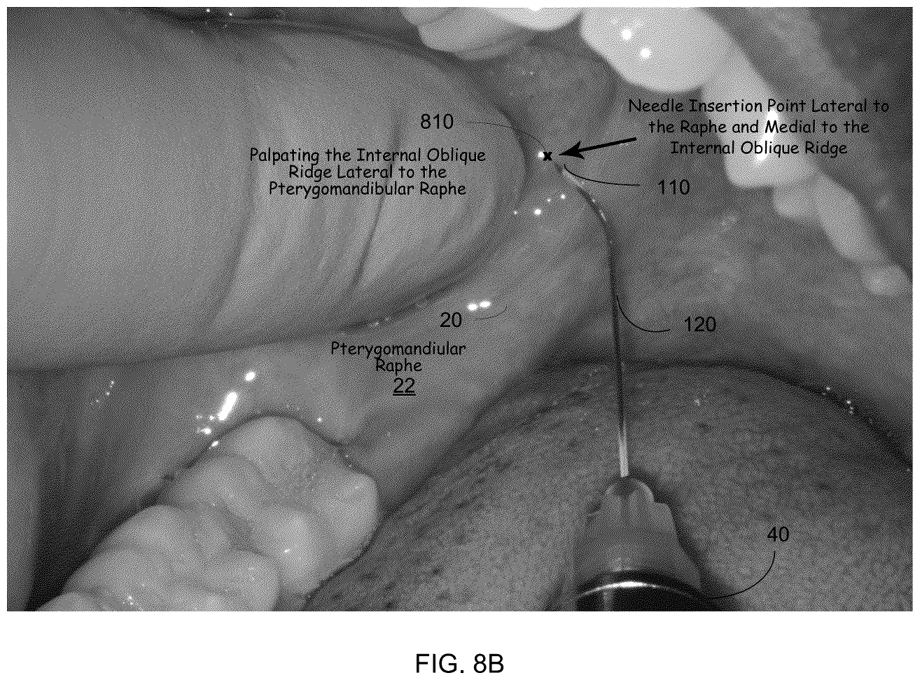

The preferred technique for using the curved needle design 100 is depicted in FIGS. 8A and 8B. The patient is reclined to approximately 135 degrees asked to open wide, as the coronoid notch 16, and the pterygomandibular raphe 22 and internal oblique ridge 20 are identified. The coronoid notch 16 is a depression lateral to the internal oblique ridge 20. The internal oblique ridge 20 is depicted by a line in FIGS. 8A and 8B. The wider the patient opens the tighter the tissues covering the internal oblique ridge 20 are stretched. The tight tissues over boney landmarks allow for better manual palpation of landmarks. Once the border of the internal oblique ridge 20 is identified, the clinician palpates the most medial aspect (surface closest to the midline or middle of body) where it begins to transition to the posterior surface (surface facing the back) using the palpating digit. This step alone eliminates the premature contact of the prior art needle tip 44 with bone as depicted in prior art FIG. 4A. The technique also provides a smaller, more definitive area of where needle insertion site 810 should be to hit the preferred anesthesia injection site 24. Once the palpating digit has identified the transition to the posterior surface, the insertion point is identified as a point that bisects the tip of the palpating digit and is half the distance from the pterygomandibular raphe 22 and the palpating digit as depicted in FIG. 8B.

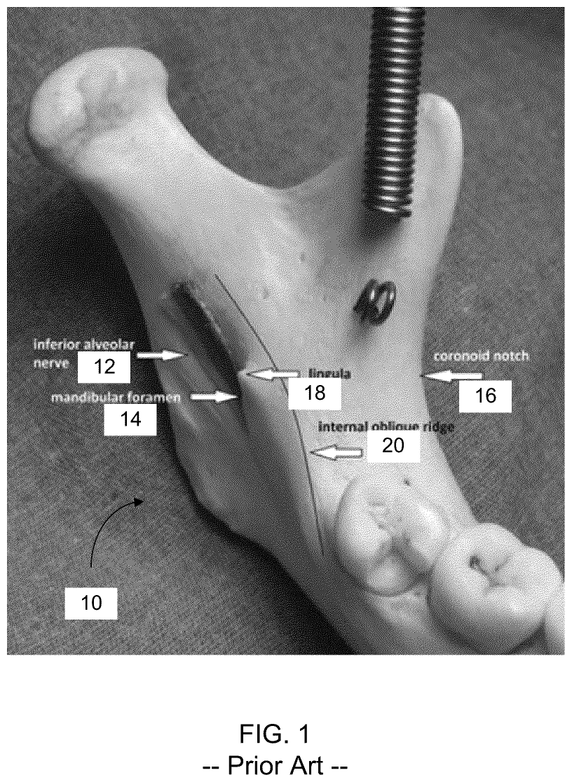

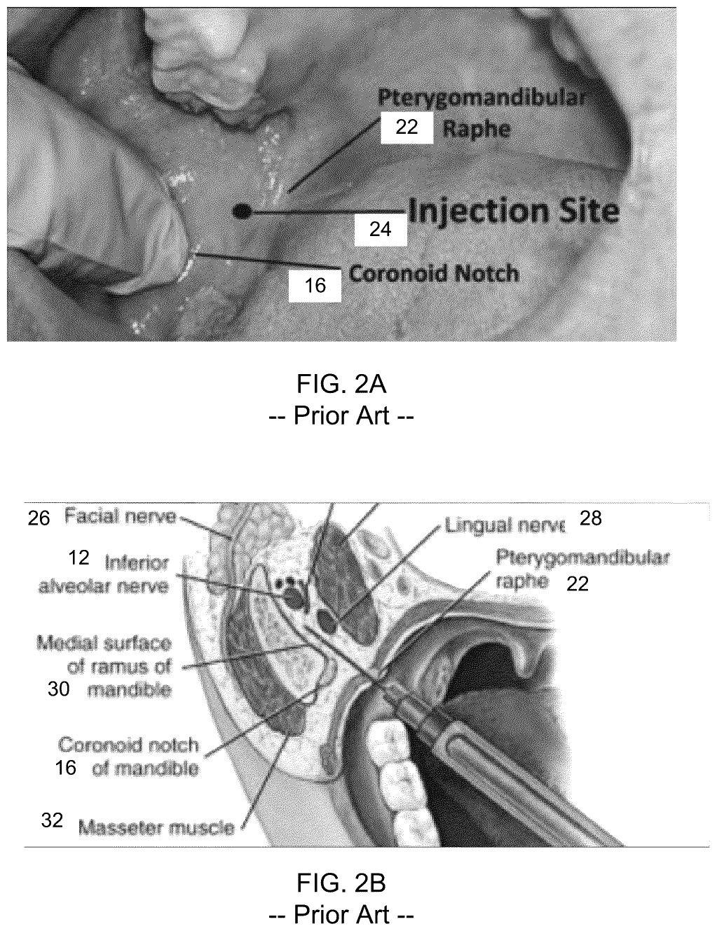

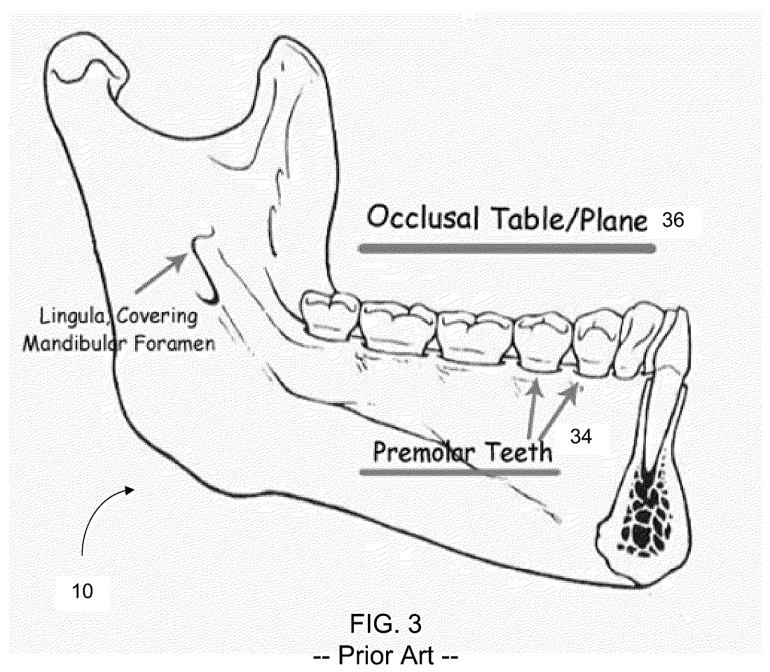

As further depicted in FIG. 8B, in the technique of the present invention the syringe barrel 40 approaches from the contralateral side of the mouth from the area of the premolars (34 in FIG. 3). The needle tip 110 is inserted at the location 810 medial (towards the midline or middle of body) to the palpated area but lateral (away from the midline or middle of the body) to the pterygomandibular raphe 22 and is simply advanced to contact bone at the medial surface of the ramus. Once bone is contacted, the needle tip is withdrawn 1-2 millimeters, needle aspiration is completed to ensure the anesthetic compound is not being injected into a blood vessel. If needle aspiration is negative (no blood withdrawn into syringe) the anesthetic compound is deposited in the pterygomandibular space adjacent to the mandibular foramen 14. If needle aspiration is positive (blood withdrawn into syringe) the needle syringe/assembly is simply rotated slightly until there is a negative aspiration and the anesthetic is deposited in the pterygomandibular space adjacent to the mandibular foramen 14. Stated another way, the needle insertion point is lateral to the raphe 22 and medial to the internal oblique ridge 20. No adjustment of the syringe barrel 40 is necessary relative to the needle tip location unless there is a positive aspiration. The curvature or angle at the needle shaft 120 deflects the needle tip 110 to the area of the mandibular foramen 14 where the anesthetic is deposited adjacent the inferior alveolar nerve 12 (depicted in FIG. 1) at the injection site 24. This step of the technique ultimately eliminates the concern of failing to contact bone due to ramus flare as depicted by the prior art needle tip 44 in prior art FIGS. 4B and 4C.

In the technique of the present invention the patient's internal oblique ridge 20 is palpated using the clinician's (person delivering the pharmaceutical compound) thumb or index finger, pending provider's preference. The soft tissue covering the internal oblique ridge 20 is held taught with the palpating thumb or index finger for maximum confidence of the landmark (internal oblique ridge). The needle tip 110 is inserted at the insertion/injection site 810 in the patient. Specifically, the insertion/injection site will be medial to the internal oblique ridge 20, lateral to the pterygomandibular raphe 22 and directly adjacent and centered to the palpating thumb or index finger. An axial force is applied to the syringe barrel 40. The centerline axis of the shaft at the needle shaft tip 110 is deflected towards the lingula via the soft tissue adjacent to the posterior surface of the needle tip 110. Adjacent to the lingula where the inferior alveolar nerve enters the mandible is the insertion/injection site 810 for the pharmaceutical compound. Axial force is applied until the needle tip contacts bone. After bone is contacted, the needle tip 110 is withdrawn approximately 1-2 millimeters, the provider aspirates (retracts the plunger) to ensure he or she is not injecting the pharmaceutical compound into a blood vessel. If there is a negative aspiration (no blood returning to the carpule) the pharmaceutical compound is injected. If the aspiration is positive (blood returning to the carpule) the needle tip is withdrawn another millimeter, aspiration is performed again, and the pharmaceutical compound is injected when there is a negative aspiration.

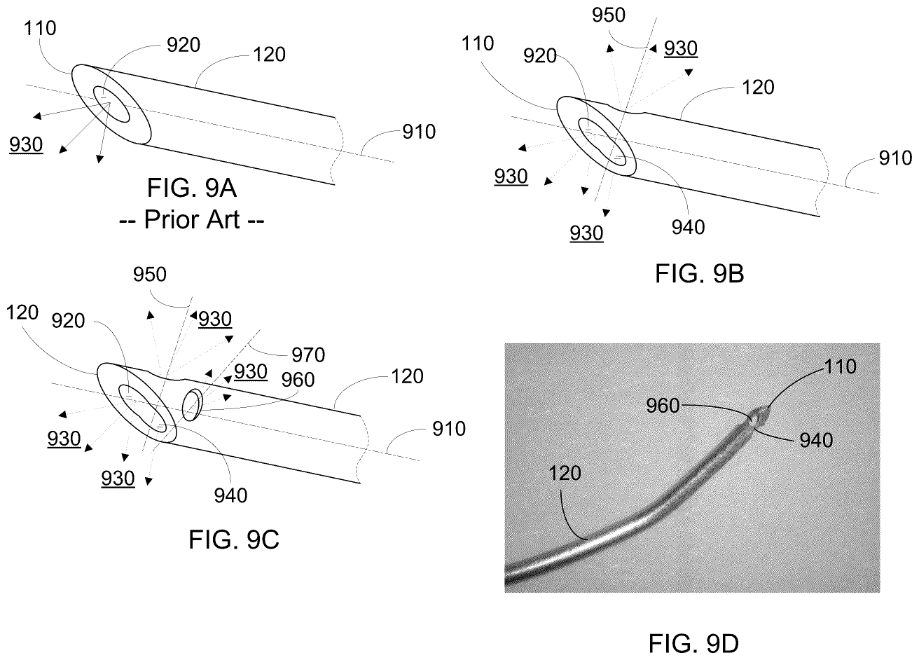

The present invention provides improvements in the design of the tip 110 of the needle shaft 120. In the prior art needle as depicted in FIG. 9A, the tip 110 is formed by a ground surface angled with respect to the centerline 910 of the needle shaft 120 to form a sharp point. The anesthetic or other pharmaceutical compound used in the procedure passes thru the needle bore 920 running the length of the needle shaft 120 at the central axis 910 of the shaft 120. The pharmaceutical compound passing thru the needle assembly is depicted as arrows 930 leaving the needle bore 920.

In an embodiment of the present invention depicted in FIG. 9B, a hole or port 940 is formed proximate to the tip 120 and passing thru both the angled surface of the tip 120 and the central bore 920 along an axis depicted as 950. The axis 950 of the hole 940 may substantially bisect the angle between the ground surface of the angled tip 110 and the central axis 910 and passes thru both side walls of the needle shaft 120 and is in fluid connection with the needle bore 920. At least one additional port is formed as the hole 940 passes thru the sidewall of the needle shaft 120. As depicted in FIG. 9B in another alternative embodiment, a second port is partially formed as the hole 940 intersects the angled face of the needle tip 110 effectively increasing the cross sectional area of the central bore 920 proximate to the hole. The ports or holes 940 are in fluid interconnection with the central bore 920 and anesthetic or other pharmaceutical compound used in the procedure passes thru the needle bore 920 and leaves the needle out both the central bore 920 and the ports 940 and is depicted as arrows 930. In other alternative embodiments, the hole or bore 940 may be offset from the needle tip 110 and not intersect the angled face of the tip 110.

In another alternative embodiment depicted in FIG. 9C, a hole or port 960 is formed proximate to the tip 120 and passing thru the central bore 920 along the axis depicted as 970. The axis 970 of the hole 960 is perpendicular to the axis 910 of the central bore 920, and is parallel with the ground surface of the angled tip 110. The hole 960 intersects central bore 920 and passes thru both sidewalls of the needle shaft 120 to form two new holes or ports 960. The ports or holes 960 are in fluid interconnection with the central bore 920 and anesthetic or other pharmaceutical compound used in the procedure passes thru the needle bore 920 and leaves the needle out the central bore 920, the ports 940, and the ports 960 and is depicted as arrows 930. As depicted in FIG. 9C, this embodiment may incorporate the hole 940 of the embodiment of FIG. 9B. In an alternative embodiment, the hole 940 of the embodiment of FIG. 9B is not present.

In yet another alternative embodiment depicted in FIG. 9D, the tip 110 of the needle shaft 120 is angled to a closed, but centered point. That tip 110 is preferably 3 mm in length. Where the tip ends and the shaft begins, a plurality of ports 940 and 960, or openings, for expulsion of anesthetic are formed into the tip 110 and shaft 120 junction. Each port is located at 90 degrees increments around the centerline of the needle. The tip 110 is continuous with the shaft 120 that extends to the hub 130. The central bore 920 of the shaft 120 and ports 940, 960 are in fluid interconnection and allow anesthetic to pass thru the shaft and out the ports at the injection site. As will be appreciated by those skilled in the art, the tip 110 may be between 1 mm and 5 mm in length. The tip may contain an additional axial opening for passing anesthetic thru the central bore 920 and out the tip 110 as in a conventional needle. The number of ports 940, 960 are preferably 4, but a single axial opening as in a conventional needle may be used, or a single or plurality of ports may be employed, or any combination thereof.

The design and location of the needle tip openings, or ports, of the embodiments of FIGS. 9B, 9C, and 9D allow for a “cloud” of anesthetic to be released radially from the axis of the needle tip at multiple locations in angles instead of a “stream” at a single angle primarily directed towards the intended anesthetic site. The anesthetic deposited in multiple radial directions provides a larger area for anesthetic deposition than a stream. The larger area of anesthetic deposition also compensates for anatomic variations between patients including the anterior/posterior location of the lingula as well as the lingula's inferior/superior location. The design also allows for a larger volume of anesthetic in the intended area of deposition as anesthetic is not deposited until after bone is contacted.

The curvature of the needle maximizes the effects of needle deflection to advantage to compensate for mandibular ramus flare and technique proficiency. Ultimately, the use of 3 landmarks, the curvature of the needle, and if included the multiple ports at the needle tip work in tandem to dramatically reduce the learning curve and makes the inferior alveolar nerve block a predictable and reliable technique for mandibular anesthesia. The curved needle shaft 120 design of the present invention also improves the tactile feedback to the practitioner as the needle penetrates tissue and contacts bone.

While there has been shown a preferred embodiment of the present invention, it is to be understood that certain changes may be made in the forms and arrangement of the elements of the curved dental needle without departing from the underlying spirit, scope, and essential characteristics of the invention. The present embodiment is therefore, to be considered as merely illustrative and not restrictive, the scope of the invention being indicated by the claims rather than the foregoing description, and all changes which come within the meaning and range of equivalence of the claims are therefore intended to be embraced therein.

What is claimed is:

1. A dental procedure for injection of pharmaceutical compound into an injection site at a posterior vertical wall of a patient mouth in an inferior alveolar nerve block performed by a clinician, the patients mouth having a internal oblique ridge, a pterygomandibular raphe, a lingula, a mandible bone, and a inferior alveolar nerve, the procedure utilizing a needle assembly affixed to a syringe, the needle comprising a needle shaft, a needle hub, a needle tip, with a central bore passing thru each, the syringe comprising a syringe barrel and a plunger, the procedure steps comprising;
palpating the patient's internal oblique ridge using the clinician's thumb or index finger, the internal oblique ridge providing a landmark for identification of an injection site;
identifying the injection site as medial to the internal oblique ridge and lateral to the pterygomandibular raphe and directly adjacent and centered to the palpating thumb or index finger;
inserting the needle tip at the identified injection site by applying an axial force to the syringe barrel, the needle shaft comprising a centerline axis and a curve therein such that the centerline axis of the needle shaft at the tip is at an angle when compared to the centerline axis of the needle shaft at the needle hub,
wherein the centerline axis of the needle shaft at the needle shaft tip is deflected towards the lingula of the patient by the axial force;
applying axial force to the syringe barrel until the needle tip contacts the bone of the mandible;
withdrawing the needle tip 1 to 2 millimeters and injecting the pharmaceutical compound adjacent the inferior alveolar nerve.

2. The dental procedure of claim 1, wherein after withdrawing the needle tip 1 to 2 millimeters, the clinician retracts the syringe plunger to ensure he or she is not injecting the pharmaceutical compound into a blood vessel, and upon verifying no blood is present, the pharmaceutical compound is injected.

3. The dental procedure of claim 2, wherein when blood is present and the needle tip is withdrawn an additional 1 millimeter, the clinician again retracts the syringe plunger, and upon verifying no blood is present, the pharmaceutical compound is injected.

4. The dental procedure of claim 1, wherein the pharmaceutical compound is an anesthetic.

5. The dental procedure of claim 1, wherein as the needle tip is inserted into the injection site the angled needle shaft centerline axis facilitates access to the injection site by avoiding physical obstacles presented by the patient anatomy to the needle assembly and syringe barrel.

6. The dental procedure of claim 1, wherein the angle between the centerline axis of the needle shaft at the tip when compared to the centerline axis of the needle shaft at the hub is between 15 and 45 degrees.

7. The dental procedure of claim 1, wherein the angle between the centerline axis of the needle shaft at the tip when compared to the centerline axis of the needle shaft at the hub is substantially 30 degrees.

8. The dental procedure of claim 1, wherein the needle shaft used in the procedure has a length between 20 millimeters to 40 millimeters from tip to hub measured along the needle shaft centerline.

9. The dental procedure of claim 1, wherein the needle shaft used in the procedure comprises a central bore between 20 and 32 gauge therein for passing pharmaceutical chemicals there thru.

10. The dental procedure of claim 1, wherein the needle shaft used in the procedure comprises a central bore between 25 and 27 gauge therein for passing pharmaceutical chemicals there thru.
